(19) United States
(12) Patent Application Publication
(10) Pub. No.: US 2022/0127560 A1
(43) Pub. Date: Apr. 28, 2022

(54) ISLAND BASED SYSTEM TO RECYCLE CO2 FROM COMBUSTION EMISSIONS

(71) Applicant: David Haberman, Delray Beach, FL (US)

(72) Inventor: David Haberman, Delray Beach, FL (US)

(21) Appl. No.: 17/510,719

(22) Filed: Oct. 26, 2021

Related U.S. Application Data
(60) Provisional application No. 63/106,556, filed on Oct. 28, 2020.

(51) Int. Cl. C12N 1/12; C02F 1/14; B01D 53/84; B01D 53/34; C25B 1/04; H01M 8/04082; H01M 10/46; C12M 1/00

(52) U.S. Cl. CPC C12N 1/12; C02F 1/14; B01D 53/84; B01D 53/346; C02F 1/727; H01M 8/04201; H01M 10/465; C12M 43/08; C02F 2201/009; C25B 1/04

(57) ABSTRACT

Provided is a method of capturing and recycling carbon dioxide from on-island combustion emissions using sustainable solar energized aquaculture of algae, including the steps of: generating electrical power from at least two renewable power producing systems, wherein the renewable power producing systems comprise at least a solar photovoltaic cell and a water turbine; and storing the electrical power in a battery array.

### CROSS-REFERENCE TO RELATED APPLICATIONS

This patent claims the benefit of U.S. Provisional Patent Application Ser. No. 63/106,556, filed 28 Oct. 2020, titled Island Based System To Recycle CO2 From Combustion Emissions. The entire content of this application is hereby incorporated by reference for all purposes.

### BACKGROUND

### 1. Field

The present disclosure relates generally to the use of hydrogen to recycle products of emissions, and more specifically to use hydrogen created by ocean current powered electrolysis to mitigate carbon dioxide production and release.

### 2. Description of the Related Art

Use of combustion processes on islands for the generation of power or for the manufacturing of chemicals and manufactured goods results in large carbon dioxide (CO2) emission burdens, atmospheric pollution and residual post combustion ground and water contaminants. These various forms of pollution may include particulates, ash, and heavy metals. Islands may have a limited capacity to endure the accumulations of these pollutants until they become toxic environments. The geographic isolation also bounds the energy generation and power allocation options establishing a set of limitations on energizing resources.

Islands may have an assured access to water which may be used in multiple roles within the configuration of a sustainable system: as a feedstock, a multi-process venue, a power source and as a thermal stabilization resource. Existing systems are dependent on combustion processes that are overly costly and environmentally unsound. There is a need to for a system that networks several enabling technologies into a system with unique control methods to optimize the value potential of this access to water. There is a further need to mitigate the effects of combustion emissions in order to decrease carbon emissions and overall lifecycle pollution.

### 3. Summary

The following is a non-exhaustive listing of some aspects of the present techniques. These and other aspects are described in the following disclosure.

Some aspects include a method of capturing and recycling carbon dioxide from on-island combustion emissions using sustainable solar energized aquaculture of algae, including the steps of: generating electrical power from at least two renewable power producing systems, wherein the renewable power producing systems comprise at least a solar photovoltaic cell and a water turbine; storing the electrical power in a battery array; receiving carbon dioxide from one or more source of emissions; inputting the carbon dioxide into one or more algae cultivators; feeding the one or more algae cultivators with nutrients derived from a waste source of on-island agriculture; controlling operational parameters of the one or more algae cultivators using a controller, wherein: the controller is configured to be powered by the battery array; and the operational parameters comprise: temperature; humidity; and pH; and detecting and responding to contaminations in the one or more algae cultivators using a set of countermeasures triggered by multi-modes sensor array, wherein the responding comprises a compartmentalization of a compromised subsection of the one or more algae cultivators with direct application of biologic neutralizers.

Some aspects include a tangible, non-transitory, machine-readable medium storing instructions that when executed by a system cause the processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a block logical and physical architecture diagram showing an embodiment of capturing and recycling carbon dioxide from on-island combustion emissions using sustainable solar energized aquaculture of algae in accordance with some of the present techniques;

FIG. 2 is a flowchart showing an example of a process of capturing and recycling carbon dioxide from on-island combustion emissions using sustainable solar energized aquaculture of algae in accordance with some of the present techniques; and

FIG. 3 shows an example of a computing device by which the above-described techniques may be implemented.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventor had to both invent solutions and, in some cases just as importantly, recognize problems overlooked (or not yet foreseen) by others in the field of pollution control on small contained islands. Indeed, the inventors wish to emphasize the difficulty of utilizing the types of energy inputs described herein to convert renewable ocean current energy into an ability capture carbon in a manner that is economically viable and environmentally sound. Further, because multiple problems are addressed, it should be understood that some embodiments are problem-specific, and not all embodiments address every problem with traditional systems described herein or provide every benefit described herein. That said, improvements that solve various permutations of these problems are described below.

In some embodiments, a system 100 of capturing and recycling the carbon dioxide from on-island combustion emissions is disclosed using sustainable solar energized aquaculture of algae.

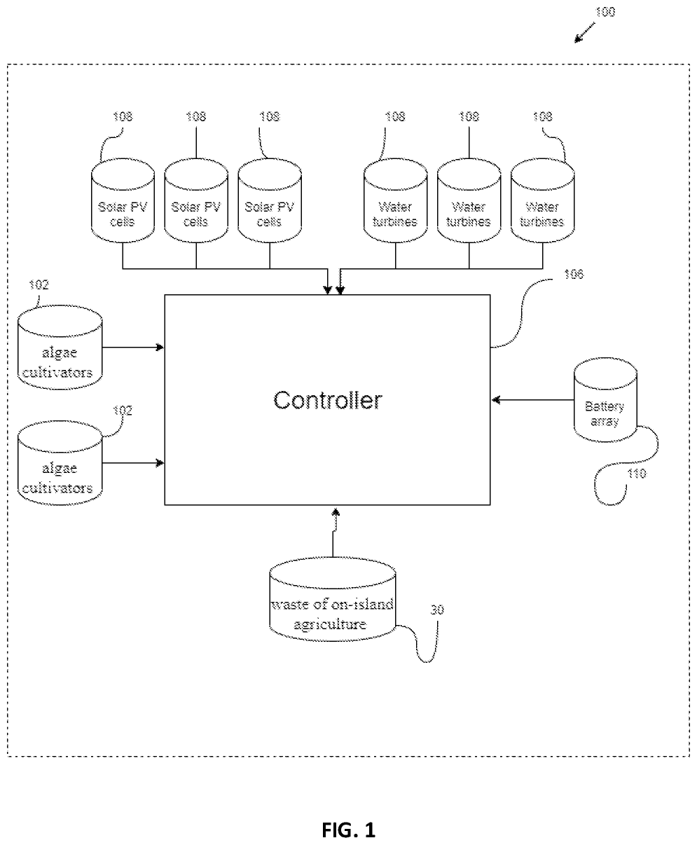

FIG. 1 is a schematic block diagram of an example of a system 100 of capturing and recycling the carbon dioxide from on-island combustion emissions is disclosed using sustainable solar energized aquaculture of algae, in which the present techniques may be implemented. A variety of different modules and computing architectures are contemplated. In some embodiments, some or all of the components of the system 100 may be hosted by different entities. In some embodiments, the system 100 and the components thereof may be implemented as a monolithic application, for instance, with different illustrated components implemented as different software modules or processes that communicate with one another, for instance via function calls, or in some cases, some or all of the components may be implemented as different processes executing concurrently on a single computing device. In some embodiments, some or all of the illustrated components may be implemented as distinct services executing on different network hosts that communicate with one another via messages exchanged via network stacks of the respective hosts, for instance, according to application program interfaces of each of the distinct services. In some embodiments, some or all of these services may be replicated, for instance, behind load balancers, to afford a relatively scalable architecture, in some cases, with elastic scaling that automatically spins up or down new instances based on load.

In some embodiments, the system 100 of capturing and recycling the carbon dioxide from on-island combustion emissions may include the steps of inputting carbon dioxide from emissions of one or more algae cultivators 102 and feeding the algae cultivation nutrients derived from the waste of on-island agriculture 104.

In some embodiments, a controller 106 may be used to manage the direct current from photovoltaic panels 108 and the alternating current from submerged water turbines 108 and store the electrical power in a battery array 110 and distribute the power to the system loads-both direct current DC and alternating AC.

In some embodiments, the controller 106 may monitor and adjust the algae cultivation water chemistry's pH using carbon dioxide. The controller may further monitor the thermal environment of the cultivation to assure optimum conditions (e.g., temperature, pH, pressure, humidity, etc.) for growth by direct temperature monitoring of cultivators and automated implementation of a set of thermal compensation measures.

In some embodiments, the controller 106 may use a custom harvesting mechanism to off-take mature algae while preserving both the baseline culture and the physical integrity of the lipid containment in the harvested alga. The controller may further detect and respond to culture contaminations using a set of custom countermeasures triggered by multi-modes sensor array which include a compartmentalization of a compromised sub section of the cultivation environment with direct application of biologic neutralizers including hot water and/or ozone.

In some embodiments, the system 100 may use the oxygen from solar energized electrolysis as a direct process amplifier and accelerator to aerobic digestion of agricultural waste and other biomass inputs optimized within the dynamic control conditions of asynchronous oxygen consumption of a biologic decomposition function and asynchronous electrochemical oxygen production function.

In some embodiments, the system 100 may use the oxygen from solar energized electrolysis as a direct process feedstock into the synthesis of green chemistry products; including herbicides, pesticides, coatings, lubricants, colorants, biofuels, materials for human consumption (e.g. nutraceuticals), sacrificial materials (e.g. organic catalysts), and animal/fish feed.

In some embodiments, the system 100 may use the hydrogen from solar energized electrolysis in a circuit with proton exchange membrane (pem) fuel cells which output reliable direct current electricity to critical system functions which require high quality electrical service such as instrumentation, safety sensors, data control, telemetry. The hydrogen fuel cell application can provide the load following pem reaction which outputs maximum power in response to the load demand. The instrumentation demands of this system using multiple biologically driven subsystems may have time domain variabilities which are imprecise. The pem fuel cell reliability may compensate in real time for these power load shifts.

In some embodiments, the system 100 may use water as a platform host and operational venue for the purpose of thermally stabilizing the heat sensitive system components, including algae cultivators, floating photovoltaic modules, green chemistry processes and heat exchangers.

In some embodiments, the system 100 may use the water heated by solar irradiance to clean and maintain the photovoltaic panels. This may be an automated cleaning process that is triggered by a custom set of sensors which detect if the panels optics are obscured by debris.

In some embodiments, the system 100 may use the controller 106 to collect relevant data for the analysis of the carbon tracking and accounting of all system inputs and output. This accounting may be converted into blockchain format for security reasons and to assure audit integrity. Controller 106 may utilize input metrics to derive carbon avoidance value units which may be reported as carbon credits within the terms of reference of applicable carbon markets; both voluntary and regulated. This information may be communicated by transponder to off-site via telemetry controls.

In some embodiments, the system 100 may collect and isolate inorganic contaminants from non-pure water sources, combustion emissions or inter-stage green chemistry processes.

In some embodiments, the system 100 may include a system health monitoring and fault/failure mitigation, specifically defining a sequence of adjusting system 100 and subsystem operational modes to preserve critical throughputs, ensure no environmental pollution and protect the integrity of capital equipment

In some embodiments, the system 100 may use pressurized carbon dioxide (CO2) to periodically flush and clean water and gas networks.

In some embodiments, the controller 106 may include an analytical tracking and audit function whereby the system operational parameters are compared with a dynamic model of the system metrics to quantify the cost ownership in real-time and provide that data to the fiscal performance tracking, export/import offset credits, special environmental incentives and tax credit determinations.

In some embodiments, the system 100 may use CO2 as a thermal coolant reservoir in selected system network positions.

In some embodiments, the controller 106 may provide sustainable agricultural ecosystem by monitoring the inputs of soil amendments, herbicide, and pesticide, and preventing the addition of any genetically modified organisms, inorganic chemicals or petrochemically derived compounds. The systemic definition of sustainable agricultural ecosystem may include the drainage and output functions of plants grown intentionally. By using soil amendments, herbicides, pesticides derived from local life systems native to the specific island ecosystem, the conduct of specialized agricultural operations may be defined as sustainable and the carbon budget of that agriculture may be quantified reliably.

In some embodiments, the system 100 may use CO2 as a critical resource which feeds proteins, lipids and starches into multiple product derivation streams (Green Chemistry). The use of ocean dynamics (e.g. submerged turbines) and solar irradiance may energize a system that synergistically couples the aquaculture of algae with the agricultural yield potential of a limited land footprint. The centralized control of the system energy exchange may empower a prioritization of power supply to both loads that use direct current and separately loads that use alternating current.

In some embodiments, floating platforms may support the use of the water as a host for power generation and provide thermal stabilization to minimize the use of power to cool active subsystems.

In some embodiments, aerobic digestors may actively decompose a broad variety of organic matter with the use of additional oxygen. The resultant decomposition may be used as nutrients to support the carbon recycling and may be handled mechanically using gravity and density separation techniques.

In some embodiments, algae cultivation may be accomplished using a variety of configurations; both open and closed bioreactor categories, which support the operational objectives of: the scale of maximizing the throughput capacity, the purity of the constituent derivatives and the functional reliability.

In some embodiments, alkaline electrolysis may be used to split water into oxygen and hydrogen.

In some embodiments, submerged wind turbines are used to translate ocean dynamics into electricity.

In some embodiments, blackbody and optical concentrators may be used to generate heat and hot water from solar irradiance.

In some embodiments, the controller 106 may have multiple different layers of monitoring The first layer of control may be centralized control of the system 100 functions that resides at the controller, which may perform some or all of the following operations:

A. Administer and mediate the inputs and outputs of the subsystems (e.g., modules, instruments, and machineries connected to the controller) to optimize outputs within the definitions of normal modes of operation;

B. Host the health monitoring functions to collect instrumentation data which detects faults and failures using an active comparator with a parametric model of the system 100;

C. Hosts the data management and telemetry communications to support the functioning of remotely accessible dashboards, cloud data storage and configuration management;

D. Interfaces with the battery based capacitance, battery and charger management functions;

E. Hosts the carbon tracking and accounting function;

F. Hosts the logistics control functions including the repair and maintenance logs; and

G. Hosts the configuration management and change control logs.

In some embodiments, the second layer of control may be distributed functional control at the subsystem level which includes modes of operation and fault/failure recoveries.

In some embodiments, the third layer of control may be automated functions that are hardwired into the system 100, such as pressure relief devices and security and firm triggered actions.

In some embodiments, the fourth layer of control may be exception human controls including emergency stops.

In some embodiments, the system may be automated and non-man tended. It may consist of all or a subgroup of the following subsystems:

A. Renewable power generation from solar irradiance and ocean dynamics;

B. Controller with power capacitance and electrical service distribution;

C. Thermal management, including heating and cooling;

D. Water management, including water treatment;

E. Electrolysis and product gas management of oxygen and hydrogen;

F. Output utilization optimization which monitors and prioritizes the system loads (uses of output);

G. Carbon tracking and accounting;

H. Cultivation, harvest and derivations of algae;

I. Control of input gas from combustion emissions handling, CO2 Separation and balance of plant;

J. Green Chemistry Processes (the uses of the system outputs to manufacture end products); and

K. Aerobic digestion and nutrient handling.

In some embodiments, the solar irradiance is used to directly excite the photovoltaic generation of direct current electricity. Photovoltaic panels may be mechanically mounted in a floating array in a manner that maximizes the use of available water surface to stabilize the exposure the solar irradiance, minimize wave impacts and use the host water body as a heat dump.

In some embodiments, the controller 106 may be a centralized master function which mediates and administers the inputs and functions of distributed controllers resident in the subsystem locations and enclosures. This controller 106 may support the use of specialized functions such as health monitoring, telemetry, and data storage. The controller may have a closely coupled battery management computer, charger and battery array.

In some embodiments, the evaporative distillation of salt water and/or grey water is accomplished in a primarily passive system which uses solar heat to vaporize the source water in a mechanical structure that directs the water vapor to a collection point while separately collecting the contaminants including salt for deposition into the contaminant depository for disposal.

In some embodiments, the blackbody solar heat absorption for water heating may raises water temperature to a sufficient temperature within a modest exposure footprint sufficient that the heated water may be used in a cleaning role for the surfaces of the optical concentrators and the photovoltaic panels.

In some embodiments, the optical concentrator of solar irradiance may achieve a high heat of water passing through a directional transparent tube located at the focal point of the solar irradiance. This very hot water may be stored in a dedicated buffer and achieves a high enough level of purity that it meets the standards as a feedstock for electrolysis.

In some embodiments, multiple configurations of both open and closed bioreactors are hosts of the large scale living aquaculture that absorbs the CO2 gas. These cultivators may have different designs based on the algae type and the optimizations targeted for the harvest which include oils starches and/or proteins. The cultivators may be equipped with selective and adaptive harvest mechanism which collect mature algae at the appropriate point in their growth cycle.

In some embodiments, algae harvesting may be performed mechanically to prevent shock and vibrations from damaging the algae crop by causing premature release of lipids from the algae being harvested.

In some embodiments, a distributed electronic ledger may be utilized for purposes including ensuring the integrity of data related to carbon accounting. The distributed ledger may include blockchain technology, to create a secure ledger that includes a record of the events, including events related to carbon tracking, or transactions that occur within the disclosed system or method.

In some embodiments, a method of capturing and recycling the carbon dioxide from on-island combustion emissions is disclosed using sustainable solar energized aquaculture of algae, comprising the steps of: inputting carbon dioxide from emissions of one or more algae cultivators; feeding the algae cultivation nutrients derived from the waste of on-island agriculture; controlling a mechanically de-coupled system by the use of a smart controller, said smart controller configured to accept direct current from one or more photovoltaic panels, said smart controller further configured to accept alternating current from submerged water turbines, storing electrical power in a battery array; distributing power to the system loads as direct current (DC) and alternating (AC); controlling the algae cultivation water chemistry's pH using carbon dioxide; assuring the thermal environment of the cultivation is optimum for growth by direct temperature monitoring of cultivators and automated implementation of a set of thermal compensation measures; using a custom harvesting mechanism to off-take mature algae while preserving both baseline culture and physical integrity of the lipid containment in harvested alga; and detecting and responding to culture contaminations using a set of custom countermeasures triggered by multi-modes sensor array which include a compartmentalization of a compromised subsection of the cultivation environment with direct application of biologic neutralizers including hot water or ozone.

In some embodiments, through compressing techniques, hydrogen gas may be compressed into medium to and high pressure for commercial usage as commercial hydrogen gas, including multiple-hundred atmosphere pressure, and may be cooled to liquid hydrogen for storage or for use as a fuel source.

In some embodiments, a oxygen module may gather oxygen and transmit the oxygen to a collocated industrial oxygen user, including a water treatment facility, for use in aeration or other such oxygenation techniques.

In some embodiments, solar energy may be used in black body and trough water collectors to heat water and transmit the water to hot water source in fluid communication with the black body and trough collectors, which may serve as the supply to an electrolysis module.

In some embodiments, the solar heat may be used for the evaporative treatment of salt water, which produces supply for the electrolysis module. In some embodiments, heated water from the blackbody and trough collectors may be used within a high pressure spray cleaning system for photovoltaic (PV) cells to maintain high efficiency. The controller 106 may set specific recurring schedules for spray cleaning the photovoltaic cells based on fixed schedule (e.g., daily, weekly, etc.) or upon need (e.g., decreased PV efficiency)

In some embodiments, a cooling bus (e.g., heat pipes) 30 with a network of heat pipes may provide a backup thermal load if an embedded cooler trips a high temperature sensor.

Some embodiments may include the method described above further comprising the step of using electricity from the at least one of the fuel cell module and H₂ dispenser module, wherein the generating step is substantially asynchronous from the on demand electricity.

In some embodiments, the system 100 may be automated and non-man tended. In some embodiments, the system 100 may comprise a subset of modules such as a renewable power generation module, an electrical control and capacitance module, a thermal management module, a water management module, electrolysis and gas management module, output utilization (system loads) module, and a carbon accounting module.

In some embodiments, adequate situational awareness of the system performance and health may be achieved by permissive use of a dedicated controller polling a health subroutine at the box level. The system 100 may directly communicate to a cloud based information system that may be summarized on a performance dashboard available at the device level using established security protocols.

In some embodiments, the system may be implemented as designed to fail “softly” by progressing through a sequence of alerts, fault notifications tied to a compensatory set of mitigation instructions prior to proceeding to fault modes. The software which enables this logic may be resident at the box level controllers/cpu.

In some embodiments, the physical compartmentalization of functions may reflect the use of off-the-shelf equipment.

In some embodiments, the system may be packaged in environmentally shielded, weather proof shelters and containers.

In some embodiments, the calibrations may be tracked at the health monitoring system and expirations of calibrations are part of regular repair & maintenance (R&M) routines

In some embodiments, the telemetry by cellular transponder may be the baseline method to preserve box level settings, health and performance data and R&M logs. Active faults and failures are prioritized in transmission and periodic repetitions of notifications. Pending R&M activities and operational interruptions can be tied to a scheduling program to support administrative planning.

In some embodiments, gas quality may be assured by a series of purity chemical sensors located at multiple different places along piping system, and if there is a degradation trend identified then a series of alerts and notifications may be generated

In some embodiments, water quality may be assured by monitoring pH, optical quality, temperature, and selected chemistry

In some embodiments, system performance parameters may be compared in real time or substantially real time to a parametric model to identify deviations in system balance. Compensatory actions may be pre-programmed based on the extent of an detected deviations.

In some embodiments, water source may be either fresh or salt or both, and there may be separate tanks for each.

The system can be designed to use either or both salt and fresh water. In terms of salt water the filtered input line can be fed to a trough buffer tank with water pump where the water is circulated and exposed to the heat from a trough concentrator to its max thermal capacitance and directed to an closed evaporative reservoir which collects the salt and redirects the water vapor to a collection tank. That tank feeds the fresh water source inlet.

In some embodiments, the system 100 may include a carbon accounting function that may be a software program which reports analytical data to a cloud based information archive and a third party auditing function. The program receives information from the system controller, and the health monitoring system and the data stream from the system inputs and output. Its function in some embodiments may be to operate a proprietary calculus to derive the carbon footprint of the system operation and compare it to an inventory of benchmark metrics. Such techniques may include using a rate of carbon production from the renewable sources compared to non-renewables (e.g., hydrocarbons) on a per watt basis.

In some embodiments, In some embodiments, the system 100 may include a health monitoring system that may include a logic set that reflects a prioritization of protection of system devices and components. This reflects an applied weighting of the relative importance of the equipment and particularly the vulnerabilities to cascading failures or increased severity of likely outcomes of catastrophic failure modes.

In some embodiments, monitoring quality of algae cultivators in terms of contaminants, detection of regulated toxics and real-time or anticipatory notifications of propagation or distribution of improperly treated water are the high value services. In some embodiments, a carbon accounting system may be embedded within the controller 106. This system may use a set of look-up tables that use the figures of merit established for each of the modules within the system 100 as high confidence metrics for determining the carbon burden of renewably energized sources.

In some embodiments, heated water from solar hot water generator (blackbody or trough concentrator) may be used to clean and maintain PV panels using a spray triggered by a pair of custom sensors: optical degradation sensor & accelerometer both mounted on the PV bezel, which indicates if the irradiance has decreased rapidly due to optical effect interference/accumulation of optical degrading coatings and a shock/vibration notification.

In some embodiments, the solar trough concentrator may use high heat to evaporate and desalinize the input salt water to a electrolyzer.

In some embodiments, the health monitoring and telemetry function are embedded in the controller and they can recover quickly from device faults and failures because the operational mode disruptions are anticipated by on-going comparison of the system performance against a comparative model which looks for degradation trends. The reset and recovery of the gas balance of plants may be faster than the status quo because the compression cycles are pre-sequenced to avoid pressure imbalances in the gas networks from abrupt shutdowns and restarts.

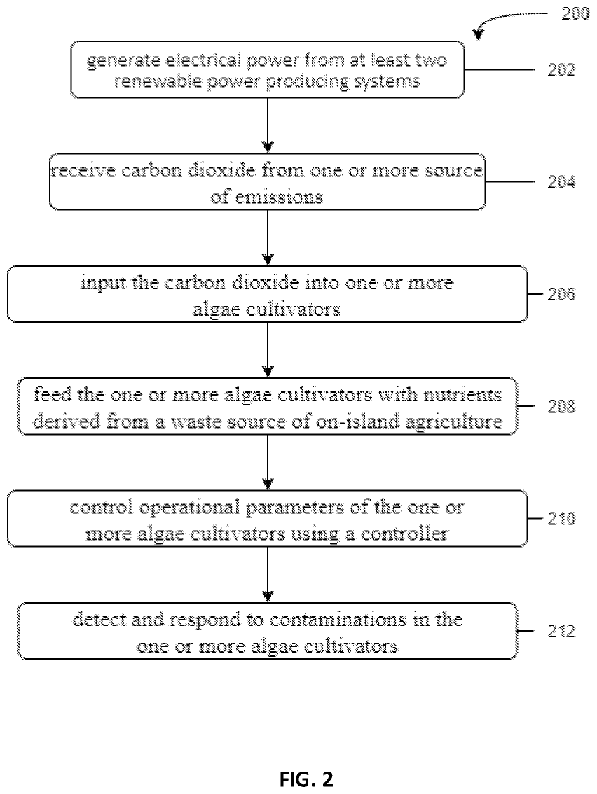

In some embodiments, the system 100 may be configured to execute the process 200 described below with reference to FIG. 2. In some embodiments, different subsets of this process 200 may be executed by the illustrated components of the system 100, so those features are described herein concurrently. It should be emphasized, though, that embodiments of the process 200 are not limited to implementations with the architecture of FIG. 1, and that the architecture of FIG. 1 may execute processes different from that described with reference to FIG. 2, none of which is to suggest that any other description herein is limiting.

In some embodiments, electrical power may be generated from renewable power producing systems such as solar photovoltaic cells and water turbines, as shown by block 202 in FIG. 2. In some embodiments, flows of carbon dioxide from one or more source of emissions may be received by the system and put into a series of algae cultivators, as shown by blocks 204 and 206 in FIG. 2. These algae cultivators may be further provided with a nutrients derived from a waste source of on-island agriculture sources, as shown in block 208 of FIG. 2. In some embodiments, the operational parameters of the algae cultivators may be monitored and controlled using a controller, as shown by block 210 in FIG. 2. Those operational variables may include temperature, humidity, and pH. In some embodiments, the controller may be configured to detect and respond to contaminations in the one or more algae cultivators using a set of countermeasures triggered by multi-modes sensor array, wherein the responding comprises a compartmentalization of a compromised subsection of the one or more algae cultivators with direct application of biologic neutralizers, as shown by block 212 in FIG. 2.

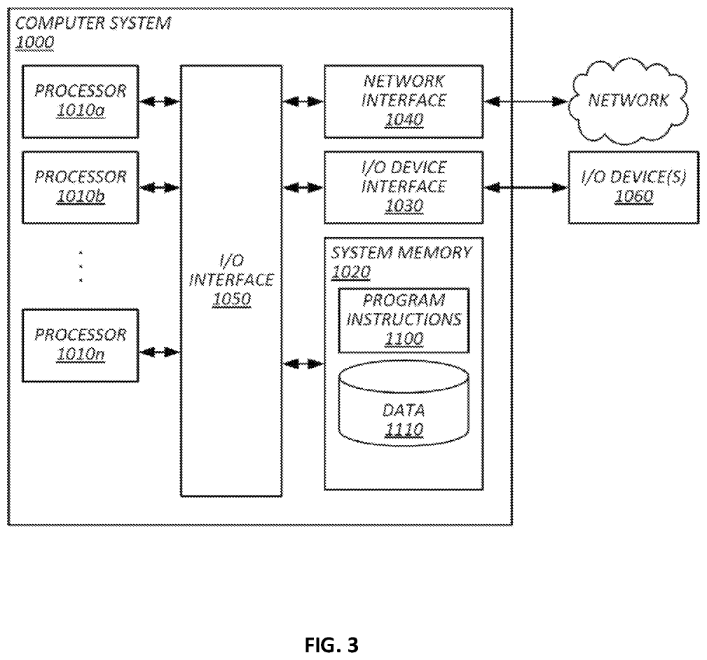

FIG. 3 is a diagram that illustrates an exemplary computing system 1000 by which embodiments of the present technique may be implemented. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating” “determining” or the like refer to actions or processes of a specific apparatus, such as a special purpose computer or a similar special purpose electronic processing/computing device. Features described with reference to geometric constructs, like “parallel,” “perpindicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation.

In this patent, certain U.S. patents, U.S. patent applications, or other materials (e.g., articles) have been incorporated by reference. The text of such U.S. patents, U.S. patent applications, and other materials is, however, only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:

1. A method of capturing and recycling carbon dioxide from on-island combustion emissions using sustainable solar energized aquaculture of algae, comprising the steps of: generating electrical power from at least two renewable power producing systems, wherein the renewable power producing systems comprise at least a solar photovoltaic cell and a water turbine; storing the electrical power in a battery array; receiving carbon dioxide from one or more source of emissions; inputting the carbon dioxide into one or more algae cultivators; feeding the one or more algae cultivators with nutrients derived from a waste source of on-island agriculture; controlling operational parameters of the one or more algae cultivators using a controller, wherein: the controller is configured to be powered by the battery array; and the operational parameters comprise: temperature; humidity; and pH; and detecting and responding to contaminations in the one or more algae cultivators using a set of countermeasures triggered by multi-modes sensor array, wherein the responding comprises a compartmentalization of a compromised subsection of the one or more algae cultivators with direct application of biologic neutralizers.

2. The method of claim 1, wherein the biologic neutralizers are hot water and ozone.

3. The method of claim 1 further comprising the step of auditing with the use of a computerized distributed ledger.

4. The method of claim 1 further comprising the step of carbon dioxide capture.

5. The method of claim 1, wherein the step of carbon dioxide capture comprises carbon capture unit, carbon recycling unit, carbon input unit, and carbon output unit.

6. The method of claim 1, wherein the solar photovoltaic cell comprises a spray triggered mechanism to wash the surface of the solar photovoltaic cell.

7. The method of claim 1 further comprising: steps for sustainable solar energized aquaculture of algae.

8. The method of claim 1, wherein a heat generated in the solar photovoltaic cell is used to evaporate and desalinize a water supply source.

9. The method of claim 1 further comprising the steps of: converting the electrical power to run an electrolyzer, wherein: the electrolyzer is in communication with a water supply source; and the electrolyzer is capable of separating H₂O into H₂ and O₂; and transporting, from the electrolyzer, the H₂ to a hydrogen storage module and transporting the O₂ to an oxygen storage module, wherein: the hydrogen storage module comprises: at least a fuel cell to provide on demand electricity; and a H₂ dispenser module; and the oxygen storage module comprises: a supply system for water treatment facility for aeration of wastewater.

10. A system for capturing and recycling carbon dioxide from on-island combustion emissions using sustainable solar energized aquaculture of algae, comprising: at least two renewable power producing systems, wherein the renewable power producing systems comprise at least a solar photovoltaic cell and a water turbine; a battery array configured to store the electrical power produced by the at least two renewable power producing systems; one or more algae cultivators; a waste source of on-island agriculture configured to provide nutrient for the one or more algae cultivators; a controller configured to monitor operational parameters of the one or more algae cultivators, wherein: the controller is configured to be powered by the battery array; and the operational parameters comprise: temperature; humidity; and pH; and a multi-modes sensor array configured to detect and respond to contaminations in the one or more algae cultivators, wherein the responding comprises a compartmentalization of a compromised subsection of the one or more algae cultivators with direct application of biologic neutralizers.

11. The system of claim 10, wherein the biologic neutralizers are hot water and ozone.

12. The system of claim 10 further comprising a computerized distributed ledger configured to audit carbon emission.

13. The system of claim 10 further comprising the step of carbon dioxide capture.

14. The system of claim 10, wherein the step of carbon dioxide capture comprises carbon capture unit, carbon recycling unit, carbon input unit, and carbon output unit.

15. The system of claim 10, wherein the solar photovoltaic cell comprises a spray triggered mechanism to wash the surface of the solar photovoltaic cell.

16. The system of claim 10 further comprising: steps for sustainable solar energized aquaculture of algae.

17. The system of claim 10, wherein a heat generated in the solar photovoltaic cell is used to evaporate and desalinize a water supply source.

18. The system of claim 10 further comprising: an electrolyzer, wherein: the electrolyzer is in communication with a water supply source; and the electrolyzer is capable of separating H₂O into H₂ and O₂; and a hydrogen storage module and an oxygen storage module, wherein: the hydrogen storage module comprises: at least a fuel cell to provide on demand electricity; and a H₂ dispenser module; and the oxygen storage module comprises: a supply system for water treatment facility for aeration of wastewater.

What is claimed is:

1. A method of capturing and recycling carbon dioxide from on-island combustion emissions using sustainable solar energized aquaculture of algae, comprising the steps of:
generating electrical power from at least two renewable power producing systems, wherein the renewable power producing systems comprise at least a solar photovoltaic cell and a water turbine;
storing the electrical power in a battery array;
receiving carbon dioxide from one or more source of emissions;
inputting the carbon dioxide into one or more algae cultivators;
feeding the one or more algae cultivators with nutrients derived from a waste source of on-island agriculture;
controlling operational parameters of the one or more algae cultivators using a controller, wherein:
the controller is configured to be powered by the battery array; and
the operational parameters comprise:
temperature;
humidity; and
pH; and
detecting and responding to contaminations in the one or more algae cultivators using a set of countermeasures triggered by multi-modes sensor array, wherein the responding comprises a compartmentalization of a compromised subsection of the one or more algae cultivators with direct application of biologic neutralizers.

2. The method of claim 1, wherein the biologic neutralizers are hot water and ozone.

3. The method of claim 1 further comprising the step of auditing with the use of a computerized distributed ledger.

4. The method of claim 1 further comprising the step of carbon dioxide capture.

5. The method of claim 1, wherein the step of carbon dioxide capture comprises carbon capture unit, carbon recycling unit, carbon input unit, and carbon output unit.

6. The method of claim 1, wherein the solar photovoltaic cell comprises a spray triggered mechanism to wash the surface of the solar photovoltaic cell.

7. The method of claim 1 further comprising:
steps for sustainable solar energized aquaculture of algae.

8. The method of claim 1, wherein a heat generated in the solar photovoltaic cell is used to evaporate and desalinize a water supply source.

9. The method of claim 1 further comprising the steps of:
converting the electrical power to run an electrolyzer, wherein:
the electrolyzer is in communication with a water supply source; and
the electrolyzer is capable of separating H₂O into H₂ and O₂; and
transporting, from the electrolyzer, the H₂ to a hydrogen storage module and transporting the O₂ to an oxygen storage module, wherein:
the hydrogen storage module comprises:
at least a fuel cell to provide on demand electricity; and
a H₂ dispenser module; and
the oxygen storage module comprises:
a supply system for water treatment facility for aeration of wastewater.

10. A system for capturing and recycling carbon dioxide from on-island combustion emissions using sustainable solar energized aquaculture of algae, comprising:
at least two renewable power producing systems, wherein the renewable power producing systems comprise at least a solar photovoltaic cell and a water turbine;
a battery array configured to store the electrical power produced by the at least two renewable power producing systems;
one or more algae cultivators;
a waste source of on-island agriculture configured to provide nutrient for the one or more algae cultivators;
a controller configured to monitor operational parameters of the one or more algae cultivators, wherein:
the controller is configured to be powered by the battery array; and
the operational parameters comprise:
temperature;
humidity; and
pH; and
a multi-modes sensor array configured to detect and respond to contaminations in the one or more algae cultivators, wherein the responding comprises a compartmentalization of a compromised subsection of the one or more algae cultivators with direct application of biologic neutralizers.

11. The system of claim 10, wherein the biologic neutralizers are hot water and ozone.

12. The system of claim 10 further comprising a computerized distributed ledger configured to audit carbon emission.

13. The system of claim 10 further comprising the step of carbon dioxide capture.

14. The system of claim 10, wherein the step of carbon dioxide capture comprises carbon capture unit, carbon recycling unit, carbon input unit, and carbon output unit.

15. The system of claim 10, wherein the solar photovoltaic cell comprises a spray triggered mechanism to wash the surface of the solar photovoltaic cell.

16. The system of claim 10 further comprising:
steps for sustainable solar energized aquaculture of algae.

17. The system of claim 10, wherein a heat generated in the solar photovoltaic cell is used to evaporate and desalinize a water supply source.

18. The system of claim 10 further comprising:
an electrolyzer, wherein:
the electrolyzer is in communication with a water supply source; and
the electrolyzer is capable of separating H₂O into H₂ and O₂; and
a hydrogen storage module and an oxygen storage module, wherein:
the hydrogen storage module comprises:
at least a fuel cell to provide on demand electricity; and
a H₂ dispenser module; and
the oxygen storage module comprises:
a supply system for water treatment facility for aeration of wastewater.
